(19) United States
(12) Patent Application Publication
(10) Pub. No.: US 2026/0076603 A1
(43) Pub. Date: Mar. 19, 2026

(54) SYSTEMS AND METHODS FOR COLLECTING, DECODING, AND MODULATING THE HUMAN MIND

(71) Applicant: OpenBCI, Inc., Brooklyn, NY (US)

(72) Inventors: Conor Russomanno, Brooklyn, NY (US); Aaron Trocola, Brooklyn, NY (US); Sean Montgomery, Reno, NV (US); Shirley Zhang, Brooklyn, NY (US); Joseph Artuso, Brooklyn, NY (US); Eva Esteban, Brooklyn, NY (US)

(21) Appl. No.: 19/399,551

(22) Filed: Nov. 24, 2025

Related U.S. Application Data
(63) Continuation of application No. 17/528,635, filed on Nov. 17, 2021, now abandoned.
Application No. 17/528,635 is a continuation-in-part of application No. PCT/US2021/015470, filed on Jan. 28, 2021.
(60) Provisional application No. 63/114,792, filed on Nov. 17, 2020.

(51) Int. Cl. A61B 5/256; A61B 5/00; A61B 5/021; A61B 5/024; A61B 5/291; A61B 5/296; A61B 5/297

(52) U.S. Cl. CPC A61B 5/256; A61B 5/0002; A61B 5/02141; A61B 5/02427; A61B 5/291; A61B 5/296; A61B 5/297; A61B 2562/0247; A61B 2562/164; A61B 2562/166; A61B 2562/18

(57) ABSTRACT

Systems, methods, and instrumentalities associated with biometrical data collection and processing are described herein, including a uniquely designed biosensing system. The biosensing system may be implemented as a wearable headset including multiple sets of physiological sensors (e.g., electrodes) configured to collect EEG, EOG, EMG, EDA, and/or PPG signals from a user's head and/or face areas. The biosensing system may be used as a standalone system or may be coupled to a VR or AR-capable device such as a VR or AR head-mounted display (HMD) to monitor a user's physiological reactions to audio/visual stimuli.

### CROSS REFERENCE TO RELATED APPLICATIONS

This application is a continuation of U.S. patent application Ser. No. 17/528,635 filed Nov. 17, 2021, and claims the benefit of PCT/US2021/015470 filed Jan. 28, 2021 which claims the benefit of priority from Provisional U.S. Patent Application No. 63/114,792 filed Nov. 17, 2020. The above-mentioned applications are incorporated herein by reference in their entireties.

### BACKGROUND

Augmented Reality (AR) and Virtual Reality (VR) systems have unlocked new avenues for scientific research and consumer entertainment. Traditional neuroscience research practices relied on oversimplified stimuli and one-way monitoring of the human brain in unnatural situations. In contrast, AR and/or VR have enabled researchers to deliver more realistic stimuli to a subject while monitoring the subject's neural and physiological reactions. These tasks can be accomplished, for example, by placing the subject in more realistic environments and determining more natural reactions of the subject based on biometric data collected from the subject. However, currently available biosensing products that can be used to collect the biometric data all have limitations. For example, these products are generally capable of collecting only one specific type of data, and/or from a particular area of the human body, such as the forehead. As a result, users who desire to collect multiple types of biometric data from a single subject need to employ multiple such biosensing products, and even assuming the desired types of data can be obtained from these products, the users still face the challenge of integrating the data produced by the different products since the data may be in different formats, have different scales, and/or be collected using different system clocks (e.g., being tagged with different timestamps). Moreover, the currently available biosensing products also lack reliability, durability, and usability, and are often built with components that are prone to wear and tear, expensive and/or difficult to replace, or uncomfortable for a subject to wear.

Accordingly, it is desirable to improve the design of biosensing products to allow for synchronized collection of multiple data types that will increase the application range of biometric data and reduce the complexity associated with system setup, data acquisition, and data reconciliation. It is also desirable to make components of the biosensing products modular, embeddable, and/or replaceable so that user comfort may be enhanced and high quality electronics may be conveniently added or replaced without significantly increasing the complexity and cost of the products.

### SUMMARY

Described herein are systems, methods, and instrumentalities associated with biometrical data collection and processing. An example biosensing system as described herein may be a wearable headset including multiple sets of physiological sensors (e.g., electrodes) configured to collect multiple types of biometrical signals from a user's head and/or face. The types of signals may include, for example, EEG (Electroencephalography) signals, EOG (Electrooculography) signals, EMG (Electromyography) signals, EDA (Electrodermal activity) signals, and/or PPG (Photoplethysmography) signals. The biosensing system may be used as a standalone system and/or may be coupled to a VR- or AR-capable device such as a VR or AR head-mounted display (HMD) to monitor a user's physiological reactions to audio/visual stimuli.

The biosensing system may include a strap subsystem and a facepad subsystem, both of which may be equipped with multiple sensors (e.g., electrodes) configured to collect (e.g., simultaneously) the types of biometrical information described herein. The sensors or electrodes as well as other components of the strap and facepad subsystems may be designed, implemented, and/or configured to be durable, comfortable, replaceable, and compatible with various external devices or programs. The quality, accuracy, and consistency of data collection are also improved over existing systems or devices because of the unique design and techniques described herein.

### BRIEF DESCRIPTION OF THE DRAWINGS

A more detailed understanding of the examples disclosed herein may be had from the following description, given by way of example in conjunction with the accompanying drawing.

FIG. 1 is a diagram illustrating an example environment in which a biosensing system described herein may be deployed to determine the biometrics associated with an end user.

FIG. 2A is a diagram illustrating an example of a biosensing system described herein.

FIG. 2B is a diagram illustrating an example of a biosensing system described herein.

FIG. 2C is a diagram illustrating an example of a biosensing system described herein.

FIG. 2D is a diagram illustrating an example of a biosensing system described herein.

FIG. 3A is a diagram illustrating an example of a biosensing system being coupled with a head-mounted display (HMD) device.

FIG. 3B is a diagram illustrating an example of a biosensing system being coupled with a head-mounted display (HMD) device.

FIG. 3C is a diagram illustrating an example of a biosensing system being coupled with a head-mounted display (HMD) device.

FIG. 4 is a diagram illustrating an example of a strap subsystem described herein.

FIG. 5A is a diagram illustrating an example of a midline rail that may include a guide arm and/or a ribbon cable guide.

FIG. 5B is a diagram illustrating an example of a midline rail that may include a guide arm and/or a ribbon cable guide.

FIG. 6A is a diagram illustrating an example of a strap subsystem that includes multiple printed circuit boards (PCBs).

FIG. 6B is a diagram illustrating an example of a strap subsystem that includes multiple printed circuit boards (PCBs).

FIG. 6C is a diagram illustrating an example of a strap subsystem that includes multiple printed circuit boards (PCBs).

FIG. 6D is a diagram illustrating an example of a strap subsystem that includes multiple printed circuit boards (PCBs).

FIG. 7 is a diagram of internationally recognized locations of scalp electrodes.

FIG. 8A is a diagram illustrating an example of midline active electrodes that may be included in a strap subsystem described herein.

FIG. 8B is a diagram illustrating an example of midline active electrodes that may be included in a strap subsystem described herein.

FIG. 8C is a diagram illustrating an example of midline active electrodes that may be included in a strap subsystem described herein.

FIG. 8D is a diagram illustrating an example of midline active electrodes that may be included in a strap subsystem described herein.

FIG. 9A is a diagram illustrating an example of distributed active electrodes that may be included in a strap subsystem described herein.

FIG. 9B is a diagram illustrating an example of distributed active electrodes that may be included in a strap subsystem described herein.

FIG. 9C is a diagram illustrating an example of distributed active electrodes that may be included in a strap subsystem described herein.

FIG. 10A is a diagram illustrating an example of an active electrode described herein.

FIG. 10B is a diagram illustrating an example of a sabot assembly.

FIG. 11A is a diagram illustrating an example of a polymer conductive contact assembly described herein.

FIG. 11B is a diagram illustrating an example of a polymer conductive contact assembly described herein.

FIG. 12A is a diagram illustrating an example of a conductive contact assembly.

FIG. 12B is a diagram illustrating an example of a conductive contact assembly.

FIG. 12C is a diagram illustrating an example of a conductive contact assembly.

FIG. 12D is a diagram illustrating an example of a conductive contact assembly.

FIG. 13A is a diagram illustrating an example of a conductive contact assembly including a wire strain relief guide.

FIG. 13B is a diagram illustrating an example of a conductive contact assembly including a wire strain relief guide.

FIG. 14A is a diagram illustrating an example of a conductive prong described herein.

FIG. 14B is a diagram illustrating an example of a conductive prong described herein.

FIG. 14C is a diagram illustrating an example of a conductive prong described herein.

FIG. 15A is a diagram illustrating an example of a foam pad within which an active electrode described herein may be embedded.

FIG. 15B is a diagram illustrating an example of a foam pad within which an active electrode described herein may be embedded.

FIG. 16A is a diagram illustrating an example of a facepad subsystem.

FIG. 16B is a diagram illustrating an example of a facepad subsystem.

FIG. 17A is a diagram illustrating an example of a facepad subsystem.

FIG. 17B is a diagram illustrating an example of a facepad subsystem.

FIG. 17C is a diagram illustrating an example of a facepad subsystem.

FIG. 18A is a diagram illustrating an example of a facepad PCB.

FIG. 18B is a diagram illustrating an example of a facepad PCB.

FIG. 18C is a diagram illustrating an example of a facepad PCB.

FIG. 18D is a diagram illustrating an example of a facepad PCB.

FIG. 18E is a diagram illustrating an example of a facepad PCB.

FIG. 18F is a diagram illustrating an example of a facepad PCB.

FIG. 19 is a diagram illustrating an example of a photoplethysmography (PPG) PCB.

FIG. 20 is a diagram illustrating an example of a facepad subsystem described herein.

FIG. 21A is a diagram illustrating an example of a facepad subsystem described herein.

FIG. 21B is a diagram illustrating an example of a facepad subsystem described herein.

FIG. 21C is a diagram illustrating an example of a facepad subsystem described herein.

FIG. 21D is a diagram illustrating an example of a facepad subsystem described herein.

FIG. 22 is a diagram illustrating an example of a facepad electrode described herein.

### DETAILED DESCRIPTION

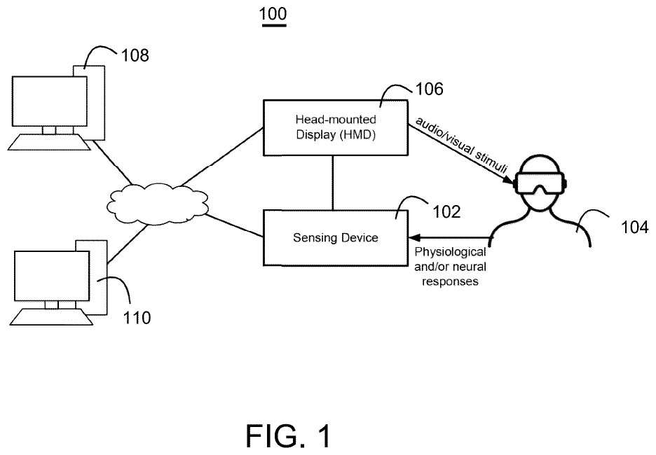

FIG. 1 is a diagram illustrating an example environment 100 where a biosensing system 102 as described herein may be deployed to determine the biometrics of an end user 104. In examples, the biometrics may include one or more of electroencephalogram (EEG) information, electrooculography (EOG) information, electrodermal activity (EDA) information, photoplethysmography (PPG) information, or electromyography (EMG) information, which may indicate the physiological reactions of the user 104 (e.g., with respect to the user's brain, eyes, skin, heart, or muscles) in response to audio and/or visual stimuli. Such audio and/or visual stimuli may be delivered to the user 104, for example, via a head-mounted display (HMD) 106 based on AR/VR content provided by a content source 108 (e.g., a content server). The user's physiological reactions may be used for various purposes including, for example, to evaluate the user's emotional response to the content, to adapt the content or create/deliver new content to the user based on the user's response, to create/enhance AR/VR experiences that allow the user to receive neurofeedback and improve his or her skills in an immersive environment, to control a device (e.g., a computer or other digital/electronic devices) based on the physiological reactions of the user (e.g., eye blinks may be used as an indication to initiate a click on a computer), and/or to conduct scientific or commercial research that may require the simultaneous collection of the data types described herein.

The biosensing system 102 may be configured to be communicatively coupled to one or more external devices such as the HMD 106, the content source 108, and/or a control device 110, and exchange information with these devices via a communication link (e.g., a wired or wireless communication link). For example, the biosensing system 102 may be configured to receive control information (e.g., operating parameters or settings for one or more components of the biosensing system) from the content source 108 and/or the control device 110, and report the biometrics (e.g., raw biometric data and/or analytics) determined based on such control information to the content source 108 and/or the control device 110. As another example, the biosensing system 102 may receive (e.g., extract) timestamp information from the HMD 106 for the audio/visual content delivered via the HMD 106 and utilize the received information to link the reactions of the user 104 to respective parts of the content delivered via the HMD (e.g., with respect to the timing of the content and the user's reactions).

While FIG. 1 illustrates a scenario where the biosensing system 102 is used in conjunction with a HMD, the biosensing system 102 may also be deployed as a standalone system (e.g., without the HMD 106) and be configured to collect biometric data from the user 104 while the user receives the audio/visual stimuli in other manners (e.g., directly from an audio/visual device and/or other stimulation devices).

The biosensing system 102 may be configured and/or calibrated via an installation (e.g., system setup) process during which the system may detect one or more external devices (e.g., the HMD 106) and provide guidance to a user of the biosensing system to connect to those devices. The biosensing system 102 may receive control information from one or more of the external devices and configure components of the biosensing system 102 (e.g., biometric sensors) in accordance with the control information so as to perform the data collection based on the user's specific needs. The control information may include, for example, operating parameters of the biosensing system 102 (e.g., types of data to be collected and/or the collection locations) and/or a destination (e.g., a cloud service or other connected devices) to transmit the determined biometrics to. The biosensing system 102 may include a communication interface and/or an application programming interface (API) that allows for transmission of the biometric data to another device (e.g., such as a 3D engine associated with the HMD 106). The API may also be configured to allow a third party program (e.g., a program written with common programming languages such Python, C++, Java, Julia, R, Matlab, and scientific protocols such as Lab Streaming Layer) to access the biometric data and enforce certain authentication rules and/or policies before the access is allowed. The biometric data transmitted by the biosensing system 102 or retrieved from the biosensing system 102 may be stored and/or processed in real time.

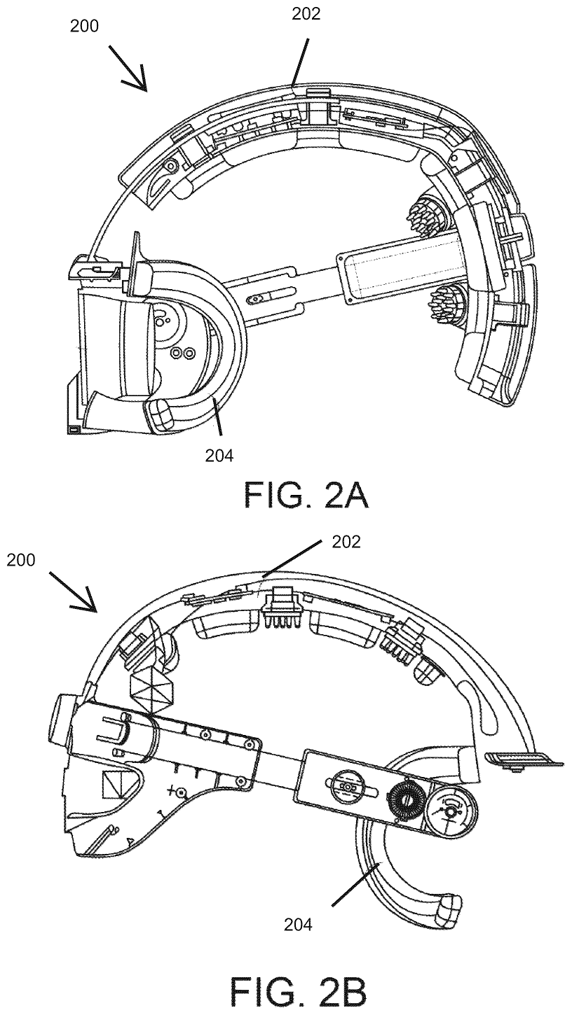

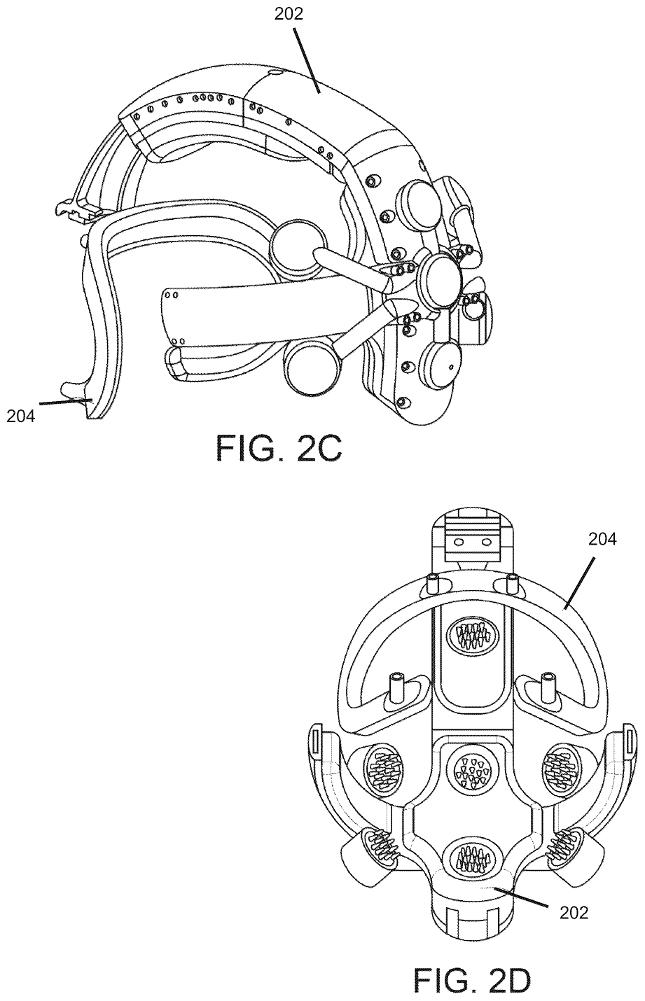

FIGS. 2A-2D illustrate an example 200 of the biosensing system described herein (e.g., the biosensing system 102 of FIG. 1). As shown in these figures, the biosensing system 200 may be configured to be head-mounted and may comprise a strap subsystem 202 and a facepad subsystem 204. The strap subsystem 202 may operate to secure the biosensing system onto a user's head, connect the biosensing system to an external device such as an HMD, and/or collect biometrical signals (e.g., EEG signals) from multiple locations of the user's scalp. The facepad subsystem 204 may operate to establish contact between one or more sensors (e.g., electrodes) of the biosensing system and corresponding areas of the user's face such that a plurality of biometrical signals may be collected from those areas to determine the user's physiological reactions to audio/visual stimuli. As described herein, the plurality of biometrical signals may be of different modalities including, for example, EEG, EOG, EDA, PPG, and/or EMG signals that indicate physiological changes of the user's brain, eyes, skin, heart, and/or muscles in response to the audio/visual stimuli.

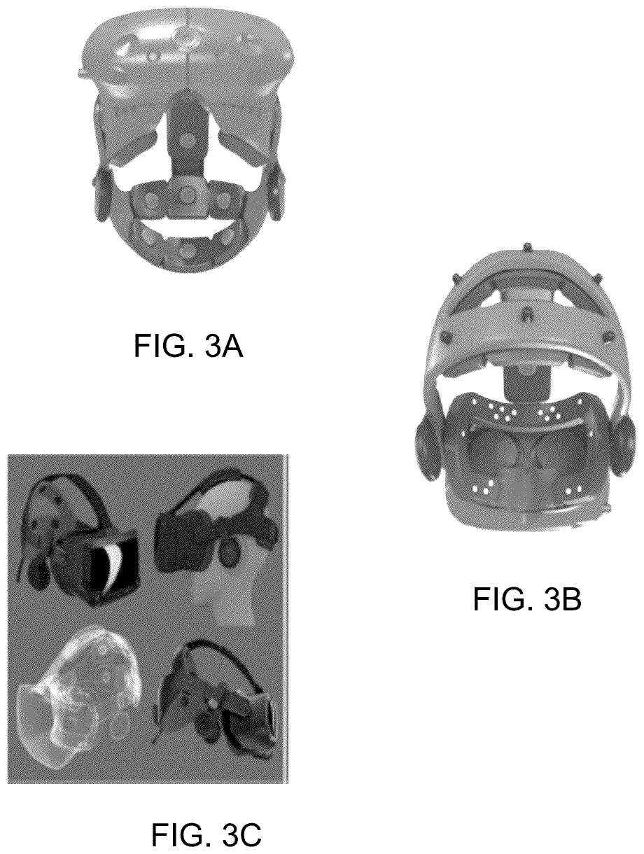

FIGS. 3A and 3B illustrate additional examples of the biosensing system from different perspectives and FIG. 3C shows examples of the biosensing system being coupled to an HMD device. The HMD device may include an AR or VR headset, and the strap and facepad subsystems of the biosensing system may include one or more custom connectors that are configured to securely and comfortably couple the strap subsystem and/or the facepad subsystem system to the AR or VR headset. The coupling of the biosensing system with such an AR or VR headset may allow for monitoring of the user's neural and physiological reactions based on more realistic audio/visual stimuli, even though the biosensing system may also be used without the AR/VR headset to determine the user's reactions based on other types of stimuli.

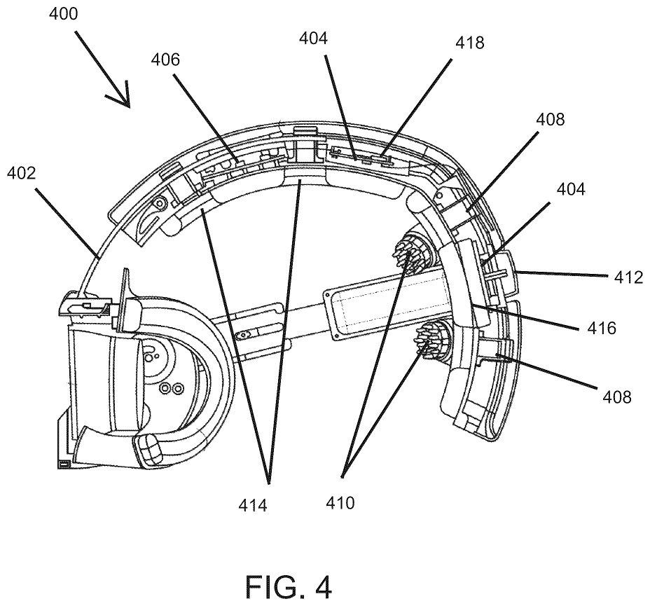

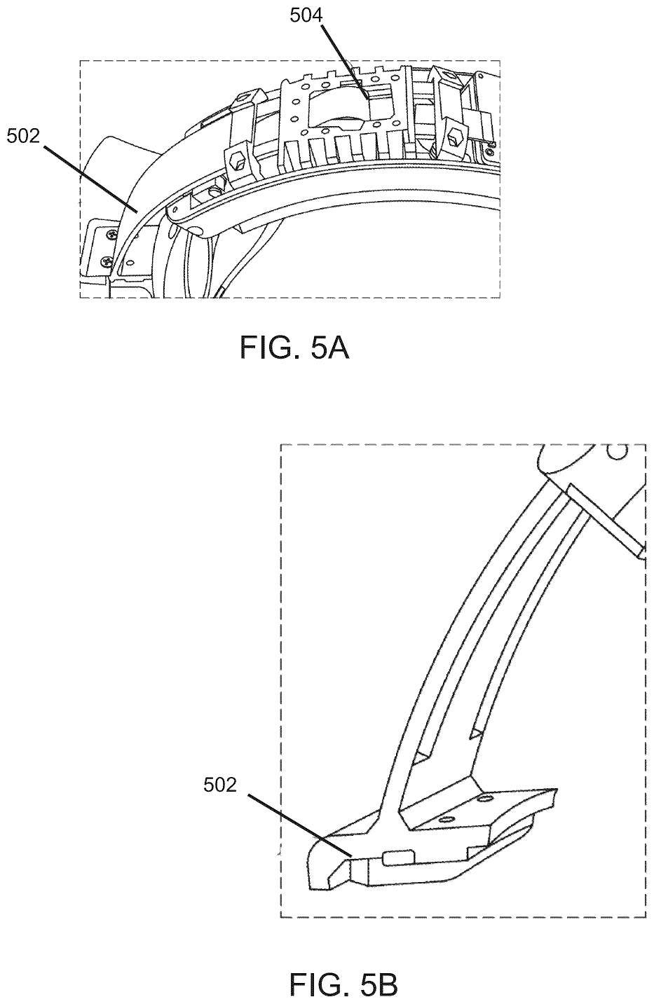

FIG. 4 illustrates an example strap subsystem 400 (e.g., the strap subsystem 202 in FIGS. 2A-2D) of the biosensing system described herein. As shown, the strap subsystem 400 may include a midline rail 402, one or more circuits 404 (e.g., one or more printed circuit boards or “PCBs”), one or more communication cables 406 (e.g., ribbon cables), one or more midline active electrodes 408 (e.g., one or more midline EEG sensors), one or more distributed active electrodes 410 (e.g., one or more distributed EEG sensors), a rear adjuster 412, one or more pressure relief pads 414, a battery 416, and/or a communication circuit 418 (e.g., a WiFi radio). As will be described below, these components of the strap subsystem 400 are configured to not only collect the biometrical data described herein, but also increase the overall comfort of the biosensing system and ensure that the system can be adjusted to fit for a variety of head sizes and/or head types. For example, FIGS. 5A and 5B illustrate examples of the midline rail 402 that may include a guide arm 502 and/or a ribbon cable guide 504 through which the one or more communication cables 406 may run. The guide arm 502 may be configured to connect (e.g., physically and electronically) the strap subsystem 400 to other parts of the biosensing system (e.g., to the facepad subsystem 204 of FIGS. 2A-D), and the guide arm may be extendable along at least a midline direction of a user's head to make the strap subsystem adjustable to different head sizes and/or head shapes. The rear adjuster 412 (e.g., a rotatable knob) may also allow a user to adjust the strap subsystem to fit the user's head size as well as to adjust the pressure applied to the user's scalp.

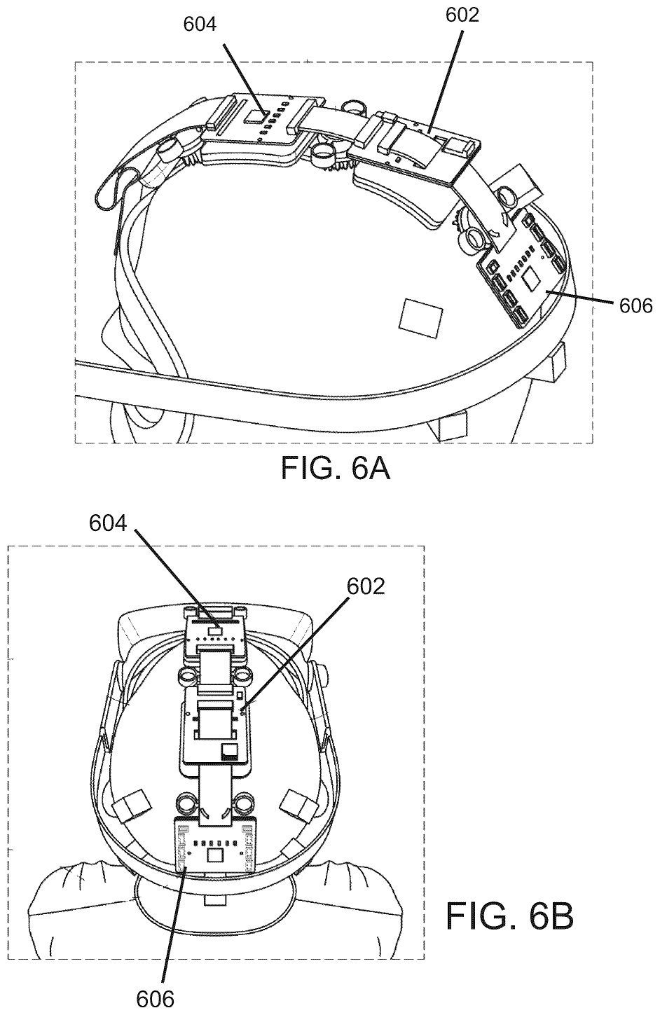

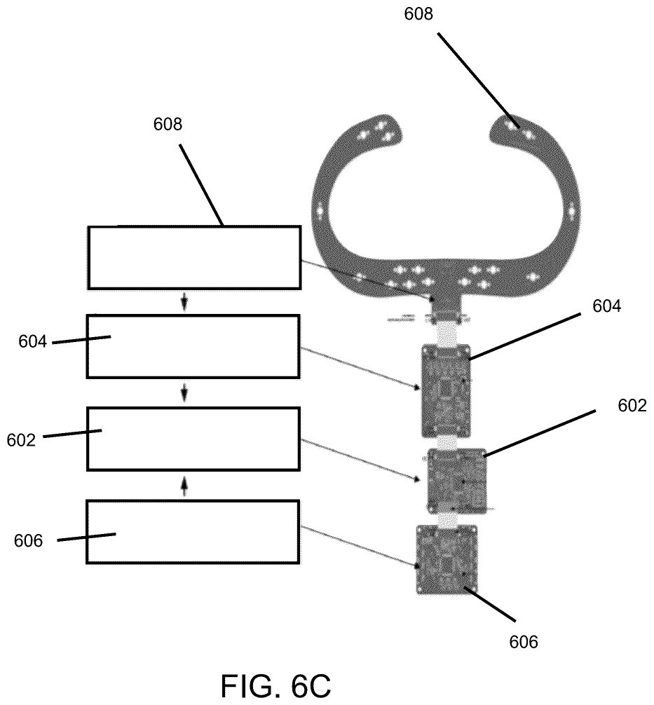

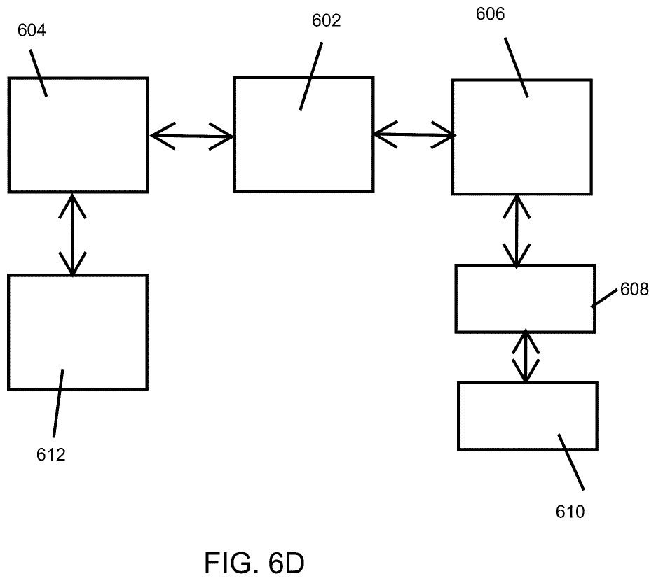

The one or more circuits 404 and/or the one or more communication cables 406 may be housed along the midline rail 402 and may include custom electrodes and/or embedded electronics configured to collect and/or process biometrical signals (e.g., EEG signals) from different areas of the user's scalp. The collection and/or processing functions may be performed with one PCB or they may be divided among multiple PCBs that are connected via the one or more communication cables 406. FIGS. 6A and 6B illustrate an example of the strap subsystem 400 that includes a main PCB 602 (e.g., a main circuit board), a first physio PCB 604 (e.g., a front physio circuit board), and/or a second physio PCB 606 (e.g., a back physio circuit board). Each of the PCBs 602, 604, and 606 may include electronics and/or programming logics for collecting and/or processing signals from different areas of the user's scalp including, for example, converting analog signals to digital signals using an analog-to-digital converter (ADC), preprocessing signals to remove noise and/or interference, tagging collected signals with corresponding timestamps, organizing signals according to user preferences, etc. For instance, the main PCB 602 may include a processing unit (e.g., a CPU, a GPU, and/or a MPU) configured to provide a system clock for the biosensing system so that biometric signals or data may be collected by the various types of electrodes (e.g., various types of sensors) described herein in a single session and then unified (e.g., fused, combined, reconciled with reference to each other) to expand the application range of the biometric data. As an example, the main PCB 602 may be configured to fuse multiple signal/data streams collected by the various types of sensors (e.g., sensor networks) into a single time series to provide a holistic view of a subject's neural and/or physiological reactions to audio/visual stimuli. The main board may also include a communication circuit (e.g., the communication circuit 418 of FIG. 4) that is configured to transmit and/or receive (e.g., wirelessly) control information and/or data to and/or from other connected devices.

The division of functionality across multiple smaller and modular boards or PCBs may provide more flexibility in the design of the strap subsystem 400 while also allowing the strap subsystem to be closely aligned with the shape of the human head, thus improving not only the sensitivity and accuracy of the signal collection, but also the overall comfort level of the biosensing system. The one or more PCBs of the strap subsystem 400 may be communicatively coupled to other circuits of the biosensing system such as one or more circuits in the facepad subsystem (e.g., a facepad flexible PCB 608 and/or a PPG circuit 610) and/or one or more active electrodes 612 of the strap subsystem (e.g., the midline active electrodes 408 and/or distributed active electrodes 410), as shown in FIGS. 6C and 6D.

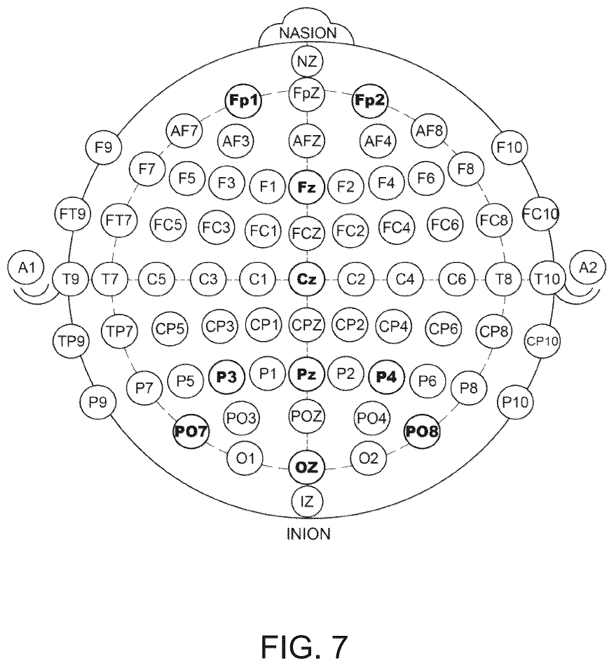

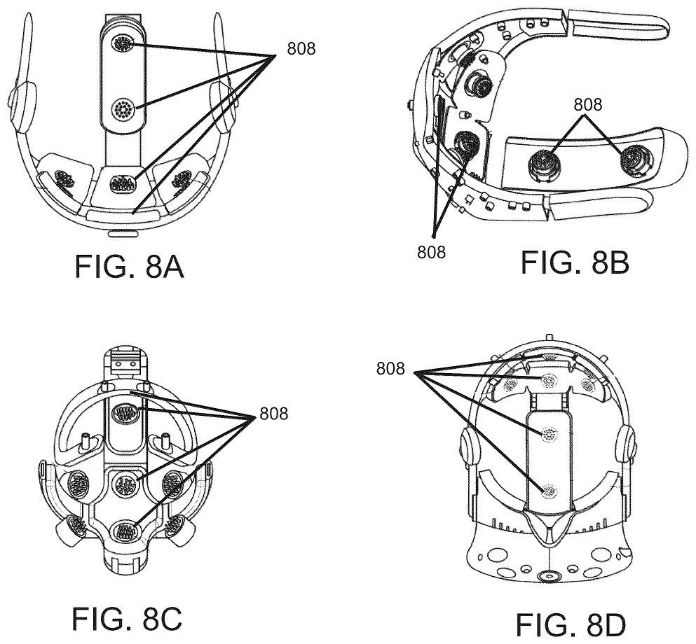

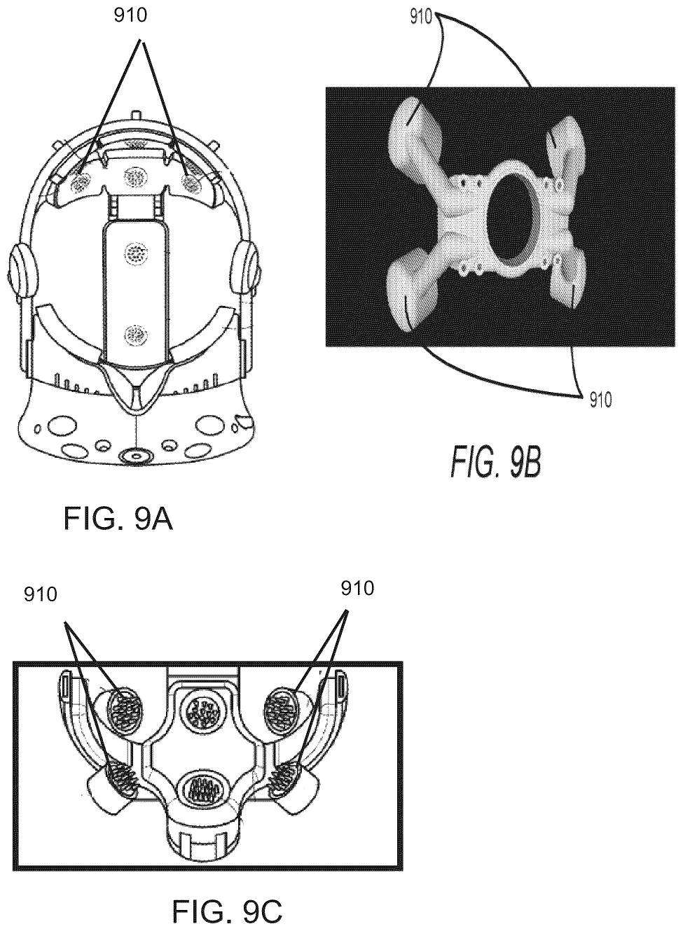

The active electrodes described herein may be configured to collect biometrical signals (e.g., EEG signals) from different areas of the user's scalp. These active electrodes may be positioned in the strap subsystem such that they may contact areas of the user's scalp in accordance with internationally recognized locations of scalp electrodes (e.g., as shown in FIG. 7) and collect signals from those areas. For example, the strap subsystem 400 may include a plurality of midline active electrodes (e.g., 4 active electrodes) that are aligned with a first set of areas (e.g., midline areas Fz, Cz, Pz, and Oz) in FIG. 7, and a second plurality of distributed active electrodes (e.g., 4 active electrodes) that are aligned with a second set of areas (e.g., occipital areas P3, P4, PO7, and PO8) of FIG. 7. FIGS. 8A-8D illustrate examples of midline active electrodes 808 (e.g., the midline active electrodes 408 in FIG. 4) that may be included in the strap subsystem. FIGS. 9A-9C illustrate examples of distributed active electrodes 910 (e.g., the distributed active electrodes 410 in FIG. 4) that may be included in the strap subsystem. As shown, the midline active electrodes 808 and the distributed active electrodes 910 may be placed (e.g., embedded) in the middle and occipital sections of the strap subsystem, respectively, and be configured to collect biometrical signals (e.g., EEG signals) from one or more areas in those sections.

The design and/or implementation of the active electrodes described herein may ensure that the electrodes make close contact with a user's scalp and be durable, replaceable, and comfortable to use. For instance, the active electrodes may be implemented using flexible conductive materials that may deform in predictable manners when pressure is applied to the electrodes (e.g., once the biosensing system is secured to the user's head and activated). As another example, the active electrodes may each include a plurality of conductive projections (e.g., combs, prongs, or spikes that may contact/engage a user's scalp) for collecting signals from multiple points of contact in and around the area where the active electrode touches the user's scalp.

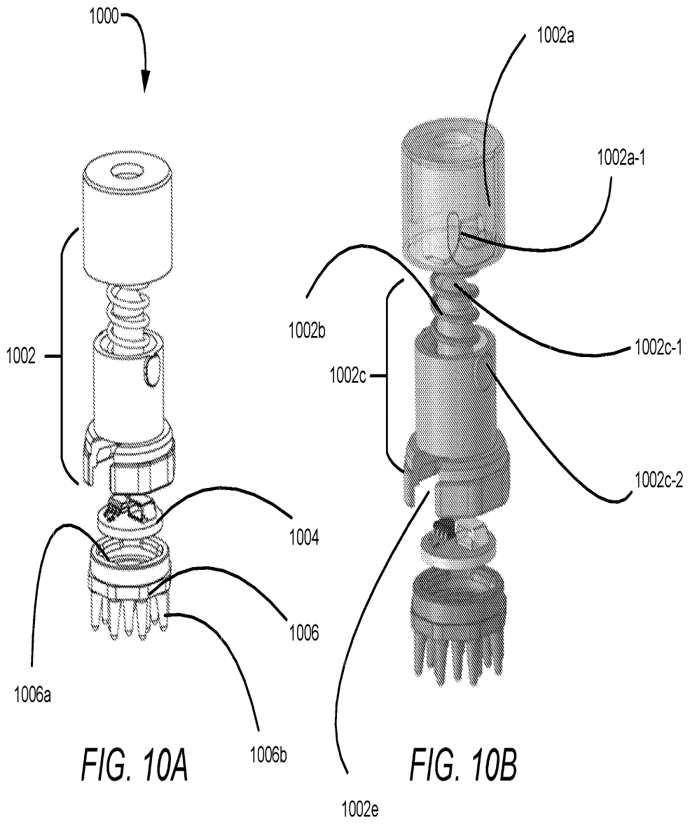

FIG. 10A illustrates an example of the active electrode 1000 (e.g., the midline active electrode 408 or the distributed active electrode 410 in FIG. 4) as described herein. As shown, the active electrode 1000 may include a sabot assembly 1002, a circuit board 1004 (e.g., a PCB), and/or a conductive contact assembly 1006. Each of these components may be designed in manners that allow them to be combined modularly and/or replaced individually. An example of the sabot assembly 1002 is shown in FIG. 10B, in which the sabot assembly 1002 is shown as including a cap 1002a, a spring 1002b, and a casing 1002c (e.g., a cylinder-shaped casing). Such a spring-loaded sabot assembly may operate to provide pressure relief to a user wearing the biosensing system described herein and/or ensure that the biosensing system be adaptable to account for differences in head size and shape between individual users. For example, the cap 1002a may be configured to orient the sabot assembly 1002 towards a user's scalp and/or to provide a backstop for the spring 1002b. The cap 1002a may also serve as a connection point between the active electrode and another device (e.g., a headset) with which the active electrode is combined. The spring 1002b may operate to provide pressure relief and ensure that the active electrode 1000 maintain close contact with the user's scalp. The casing 1002c may include a peg 1002c-1 (e.g., a post), a protrusion 1002c-2, and/or a wiring channel 1002e. There may be a first opening at the bottom of the casing 1002c that allows the combined PCB 1004 and conductive contact assembly 1006 to be fit into the bottom of the casing. There may also be a second opening along an outer wall of the casing 1002c that may serve as a channel for wiring between the PCB 1004 and a connected device. The peg 1002c-1 may be located at the center of the casing 1002c and be configured to hold the spring 1002b in place (e.g., the spring 1002b may be disposed around the peg 1002c-1). The peg 1002c-1 may extend through a hole at the top of the cap 1002a when the casing 1002c and the cap 1002a are locked together. The peg 1002c-1 may be manipulated to make fine adjustments to the position of one or more conductive prongs of the active electrodes while the electrode is in use, allowing a user to ensure that the conductive prongs extend through the user's hair and maintain close contact with the user's scalp.

The casing 1002c and the cap 1002a may be configured so that the casing 1002c may be locked into place within the cap 1002a or unlocked from the cap 1002a, for example, by fully compressing the spring 1002b and twisting the casing 1002c into a locked or unlocked position. The protrusion 1002c-2 may be located on the outside of the casing's top edge and may be configured to fit into a locking track 1002a-1 of the cap 1002a, for example, along the inside of the cap's outer wall. This locking mechanism may allow for individual components to be easily replaced, while also preventing the casing 1002c, the PCB 1004, and the conductive contact assembly 1006 from becoming detached accidentally while in use. The locking mechanism may also allow the active electrode to be combined with (e.g., fit into) another device (e.g., a headset), e.g., by inserting the cap 1002a into a receptacle included in or attached to the other device.

The PCB 1004 of the active electrode 1000 may be configured to receive the signals (e.g., analog signals) collected via the conductive contact assembly 1006 and prepare the signals for further processing by other unit(s) or component(s) of the biosensing system. For example, the PCB 1004 may be configured to apply amplification (e.g., active amplification) to the analog electrical signals collected via the conductive contact assembly 1006 before passing the amplified signals to another unit or component for processing. While the examples are described herein using active electrodes (e.g., capable of providing active amplification to the collected signals), part or all of the examples may also be implemented using other types of electrodes including, e.g., passive electrodes that may not apply amplification to the collected signals.

The conductive contact assembly 1006 may be configured to enclose the PCB 1004, for example, in a press fit bed 1006a. The press fit bed 1006a may be made of a flexible and/or conductive material such as a conductive polymer, and be shaped and/or configured to maintain close contact with the PCB 1004. In example embodiments, the press fit bed 1006a may have a raised (e.g., convex or curving outward) surface (e.g., a circular surface) at the bottom of the press fit bed that is designed (e.g., curved) to maximize the contact area between the press fit bed and the bottom surface (e.g., a metal bottom surface such as a copper surface) of the PCB 1004 when the PCB is pressed into the press fit bed. The surface of the press fit bed may flex predictably under pressure (e.g., as a characteristic of the polymer material from which the press fit bed may be made), securing the contact between the press fit bed 1006a and the PCB 1004 and increasing the number of electrons that may flow from the conductive contact assembly 1006 into the PCB 1004 when the two parts are assembled together.

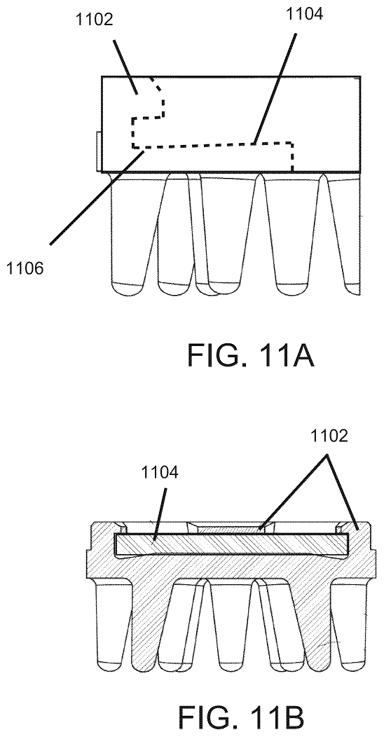

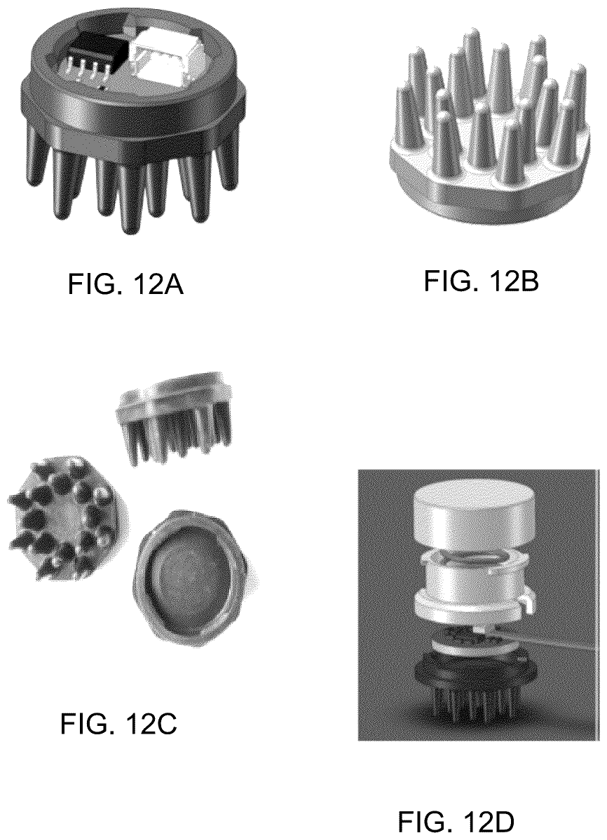

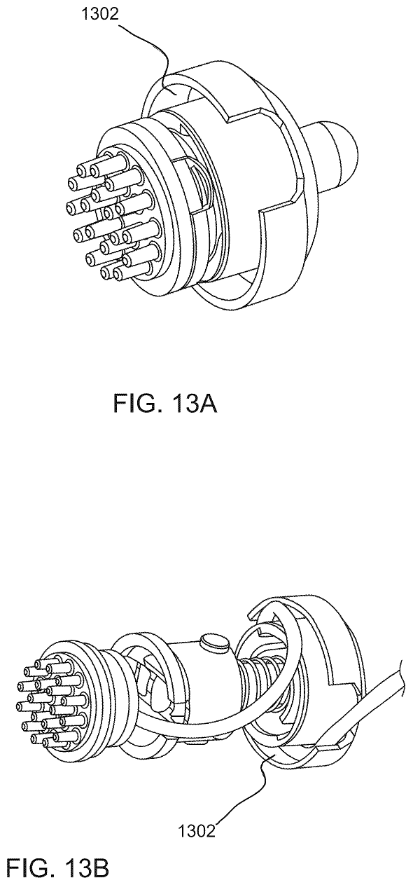

FIGS. 11A and 11B illustrate examples of the conductive contact assembly described herein (e.g., the conductive contact assembly 1006 in FIG. 10A). As shown, the conductive contact assembly may include one or more overhanging flanges 1102 located around the rim of the conductive contact assembly to hold a PCB (e.g., the PCB 1004) in place, for example, by providing downward pressure on the top surface of the PCB. This pressure may cause the scalp engagement devices (e.g., the conductive prongs described below) of the active electrode to begin to flex outward and result in the PCB being pressed into the center of the press fit bed described herein. The center of the press fit bed may include a raised, flat, and/or circular surface 1104 and there may be a downward taper 1106 (e.g., at the outer edge closest to the polymer walls) configured to give the polymer material room to flex as pressure is applied, without losing contact with the PCB (e.g., at the center of the bed). Maintaining secure contact between the PCB and the press fit bed may ensure that minimum noise caused by unnecessary movement of one or more of the components described herein not be introduced into the signals acquired via the biosensing system. FIGS. 12A-12D show additional examples of the conductive contact assembly seen from different perspectives, and FIGS. 13A-13B show examples of the conductive contact assembly including a wire strain relief guide 1302.

The conductive contact assembly described herein (e.g., the conductive contact assembly 1006 of FIG. 10) may include multiple (e.g., 16) prongs (e.g., conical protrusions 1006b shown in FIG. 10) that are capable of extending through a user's hair and making contact with the user's scalp when the conductive contact assembly is pressed against the user's scalp (e.g., while the active electrode 1000 is in use). The prongs may be made of a conductive polymer and may be arranged to allow the prongs to predictably and comfortably bend outward under pressure to ensure signal detection as well as user comfort.

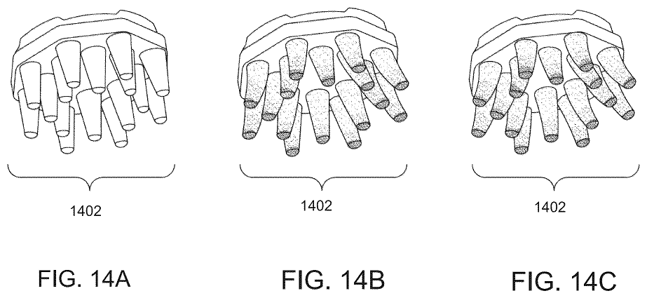

FIGS. 14A-14C illustrate examples of the conductive prongs described herein. FIG. 14A may show the example prongs 1402 under no pressure, FIG. 14B may show the example prongs 1402 under low pressure, and FIG. 14C may show the example prongs 1402 under high pressure. As shown in the examples, the prongs 1402 may be arranged into one or more concentric rings (e.g., two rings each comprising 8 prongs) around the center of the bottom of the conductive contact assembly (e.g., other non-ring type of arrangement such as arrays may also be used so long as the arrangement can accomplish the design goals described herein). In example embodiments, the center (e.g., the absolute center) of the bottom surface itself may be left open (e.g., not occupied by any prongs) to increase the overall comfort of the active electrode and/or improve the contact between the electrode and a user's scalp (e.g., the open center may account for the natural curvature of a human scalp). Not having a central prong may prevent the majority of the force/pressure from being focused through the central prong and may allow the force/pressure to be dissipated into the surrounding radial prongs (e.g., 16 radial prongs). The outer octagonal shape of the conductive contact assembly shown in the examples may ensure safe and secure fitting of the conductive contact assembly into other components or devices, while also increase the aesthetic appeal of the assembly.

Individual ones of the prongs 1402 may be configured to angle away from the center of the conductive contact assembly such that the side furthest from the center may be perpendicular (e.g., substantially perpendicular) to the bed of the conductive contact assembly and the inner edge of the prong may be at an obtuse angle with the bed of the conductive contact assembly (e.g., the exact shape of a prong may be the same as or may be different from that of other prongs). Shaping and/or angling the prongs 1402 in these manners may encourage the prongs to bend outward relative to the center of the conductive contact assembly when pressure is applied, thus preventing the prongs from folding or bending in different directions that may reduce the quality of the signals collected via the prongs. The design and/or configuration of the prongs may also ensure that the prongs maintain uniform contact with a user's scalp and be comfortable to the user's scalp. Further, the outward bending of the prongs may also enhance the contact between the press fit bed (e.g., bed 1006a of FIG. 10) and the PCB (e.g., PCB 1004 of FIG. 10) described herein since the bed is to bend as the prongs spread outwards, all while maintaining close contact with the center of the PCB, where the signals collected from the user's scalp may be transmitted to the PCB. In example embodiments, the conductive contact assembly and/or the prongs 1402 may be made of a flexible, conductive material such as silver powder in a silicone matrix, graphite in a 3D printed UV resin, and/or the like. In example embodiments, the conductive contact assembly and/or the prongs 1402 may be treated with conductive coatings, such as Ag—AgCl, to further improve the quality of signal detection and/or collection (e.g., by reducing the electrical impedance between the prongs and the user's scalp).

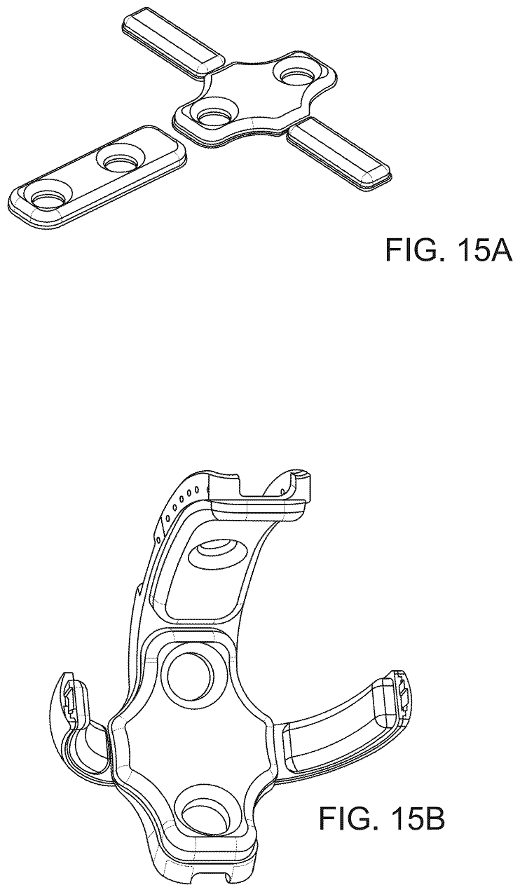

The strap subsystem described herein (e.g., the strap subsystem 400 of FIG. 4) may include one or more foam pads (e.g., memory foam pads) within which the active electrodes described herein may be embedded. FIGS. 15A and 15B illustrate examples of these foam pads. As shown, the memory foam pads may include one or more cutouts into which the active electrodes may be inserted. The foam pads may be made of materials that provide additional comfort to users of the strap subsystem.

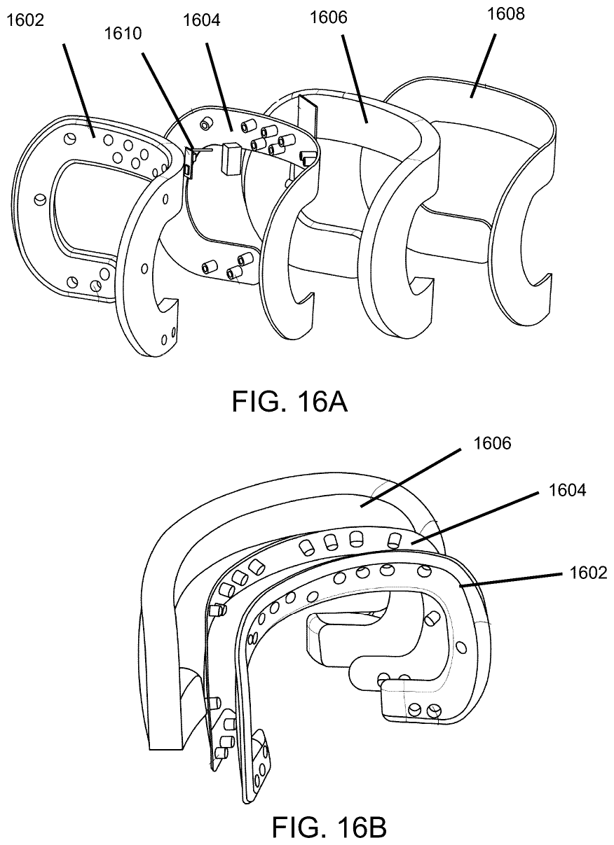

The biosensing system (e.g., the biosensing system 102 in FIG. 1 and/or the biosensing system 200 in FIGS. 2A and 2B) may include a facepad subsystem (e.g., facepad subsystem 204 in FIGS. 2A and 2B) configured to collect multiple types of biometrical signals from a user of the biosensing system. FIGS. 16A-16B illustrate examples of such a facepad subsystem. As shown, the facepad subsystem may include multiple components layered together to form the facepad subsystem. For example, the facepad subsystem may include a front pad 1602, a facepad flexible PCB 1604 (e.g., a flex facepad PCB), a mounting pad 1606, a gasket 1608, and/or a PPG circuit board 1610 (e.g., a PPG PCB comprising one or more PPG sensors) secured inside of the front pad 1602. As will be described in more detail below, including multiple layers of padding and nesting, the electronics within these layers may serve to alleviate the pressure that a user may feel when wearing the biosensing system (e.g., by distributing the pressure across multiple areas of the user's face), make the various parts modular and thus replaceable, and/or protect the electronic components of the facepad subsystem from wear and tear.

The front pad 1602 may comprise multiple sub-layers. For instance, the front pad 1602 may include a surface finish sub-layer configured to contact the user's face, an ethylene vinyl acetate (EVA) moldable foam layer located next to the surface finish sub-layer, a memory foam layer located next the EVA moldable foam layer and further away from the user's face, and/or an electromagnetic shielding layer located furthest away from the user's face and next to the facepad PCB 1604. With these sub-layers of the front pad 1602, pressure applied to the user's face may be distributed across a larger area, reducing the PSI (pound per square inch) in any specific location (e.g., without the front pad 1602, the pressure may be channeled through the metallic rigid electrodes of the facepad PCB 1604 onto the user's face). The sub-layers may also provide proper shielding between the facepad PCB 1604 and the user's face, improving both the comfort level of the facepad subsystem and the protection for the face PCB (e.g., preventing the facepad PCB 1604 from rubbing against the user's face). For example, the surface finish sub-layer may be made of a breathable material to increase the comfort of the user. Having such a surface finish sub-layer may also make it easier to wipe/clean the surface of the facepad subsystem. The EVA layer may be moldable so that it can be manipulated into a desirable shape. The memory foam layer may be compressible, which may allow the front pad to deform to different face shapes across users. The electromagnetic shielding layer may be made of a conductive material that provides electromagnetic shielding for the for the electronics of the facepad subsystem (e.g., to reduce noise or interference to the facepad sensors/electrodes).

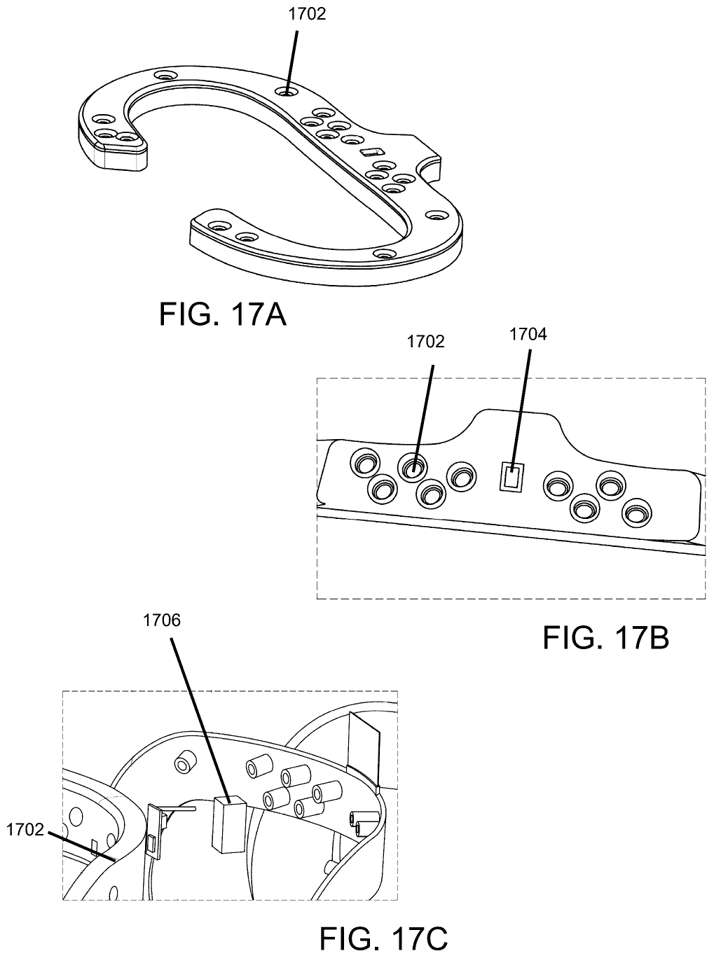

FIGS. 17A-17C show examples of the front pad 1602. As shown, the front pad 1602 may include a plurality of cutouts 1702 (e.g., 18 holes) that may allow electrodes (e.g., sensors) of the facepad PCB 1604 to pass through the front pad and contact the user's face. The front pad may also include a cavity 1704 (e.g., at the center of the front pad) and one or more support structures 1706 that may be configured to house/support the PPG PCB 1610 and/or enable one or more PPG electrodes (e.g., PPG sensors) of the PPG PCB 1610 to make secure contact with the center of the user's forehead.

Reverting to FIGS. 16A and 16B, the facepad PCB 1604 may be sandwiched between the front pad 1602 and the mounting pad 1606, and may include electronics (e.g., electrodes) and/or programming logics for collecting and processing multiple types of biometrical signals from respective areas of the user's face. The facepad PCB 1604 may be communicatively coupled to other circuits of the biosensing system (e.g., the PCB 602, 604, and/or 606 shown in FIGS. 6A and 6B), for example, via one or more communication cables (e.g., the ribbon cable 406 shown in FIG. 4) that may be attached to a guide arm (e.g., the guide arm 502 in FIG. 5) extending from the strap subsystem to the facepad subsystem. Via these communication cables, the facepad PCB 1604 may transmit the data collected and/or processed by the facepad PCB to the other circuits of the biosensing system. The facepad PCB 1604 may also be communicatively coupled to and exchange information with the PPG PCB 1610, for example, via one or more connectors (e.g., respective connectors comprised in the facepad PCB and the PPG PCB) and/or communication cables. The facepad PCB 1604 may be a flexible PCB so that it may be embedded between the multiple layers that make up the facepad subsystem. The flexible nature of the facepad PCB may allow the PCB to deform under the pressure caused by the front pad 1602 being pressed against the user's face and/or the deformation of the face gasket 1608 as the gasket flexes to accommodate the curvature of the user's face.

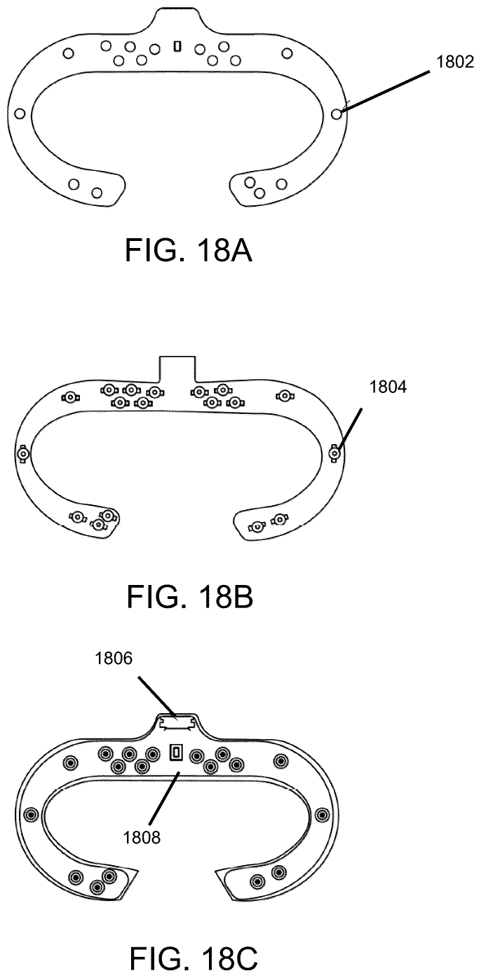

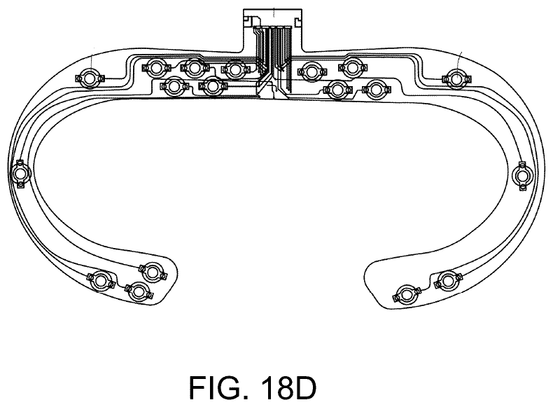

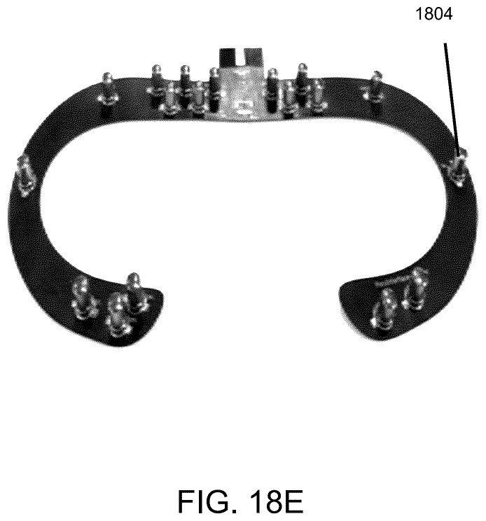

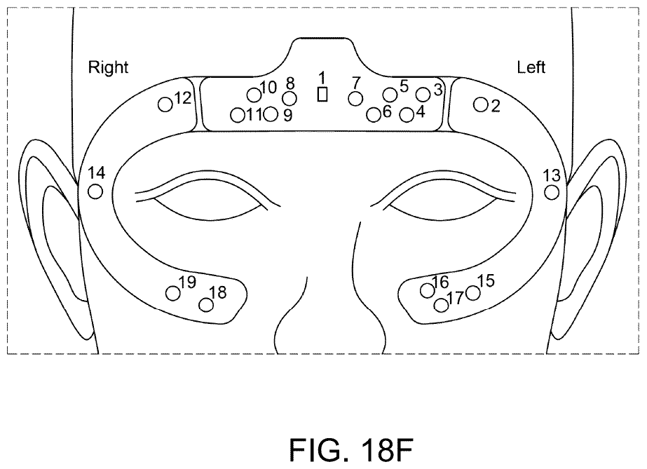

FIGS. 18A-18F show examples of the facepad PCB 1604. As shown, the facepad PCB may include one or more snap connectors 1802 (e.g., 18 female snap connectors) configured to secure respective facepad electrodes 1804 (e.g., 18 stylus electrodes or sensors) of the PCB, a connector 1806 configured to connect the strap ribbon cable described herein (e.g., the ribbon cable 406 shown in FIG. 4), and/or a connection point 1808 for a ribbon cable that connects the facepad PCB to the PPG PCB 1610 shown in FIG. 16A. The snap connectors 1802 and/or the facepad electrodes 1804 may be placed on the facepad PCB such that the facepad electrodes 1804 may contact areas of the user's face that surround the user's eyes. The facepad electrodes 1804 may be divided into groups that respectively measure and/or collect EEG, EMG, EDA, and/or EOG signals (e.g., simultaneously or in a single session). For example, two electrodes among electrodes labeled 3-6, 9-11, or 13-19 in FIG. 18F may be used to measure and/or collect EEG signals, eight electrodes among electrodes labeled 3-6, 9-11, or 13-19 in FIG. 18F may be used to measure and/or collect EMG signals, four electrodes among electrodes labeled 3-6, 9-11, or 13-19 in FIG. 18F may be used to measure and/or collect EOG signals, and two electrodes among electrodes labeled 7 and 8 in FIG. 18F may be used to measure and/or collect EDA signals. Further, one or more BIAS electrodes (e.g., electrode 2 in FIG. 18F) and/or one or more SRB2 electrodes (e.g., electrode 12 in FIG. 18F) may be included and used as reference points for evaluating the voltage measurements at the other locations. An example assignment of the electrodes based on the labeling of FIG. 18F may be as follows.

TABLE 1
Example Electrode Assignment

| Data Type | Electrode Locations |
|---|---|
| PPG | 1 |
| BIAS | 2 |
| SRB2 | 12 |
| EDA | 7, 8 |
| EEG | 5, 10 |
| EMG | 4, 6, 9, 11, 15, 16, |
| | 18, 19 |
| EOG | 3, 13, 14, 17 |

The operations of the electrodes (e.g., the operation of the electrodes measuring EEG, EMG, and EOG signals) may be configurable (e.g., using firmware of the facepad PCB) such that the electrodes may be designated to measure and/or collect any type of signals described herein (e.g., an electrode may be dynamically switched from measuring EEG signals to collecting EMG signals, or vice versa). A user may also configure other electrode settings such as a PGA gain, whether a Bias or SRB2 electrode location is to be used as a reference point in calculating the voltage data collected by a specific electrode, etc. Greater detail about the facepad electrodes 1804 will be provided below.

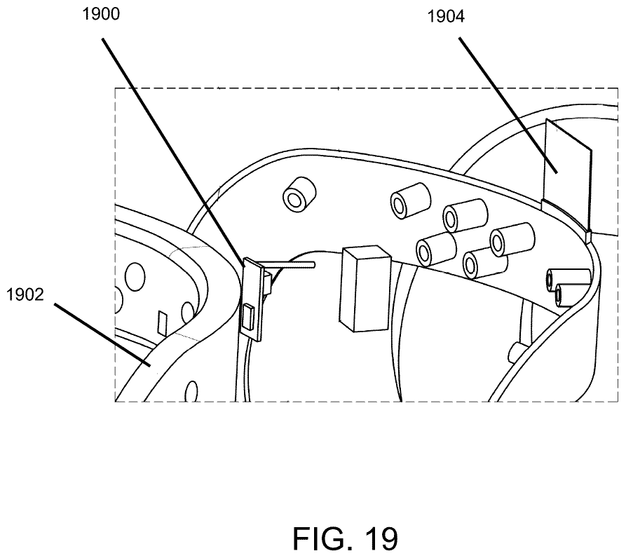

FIG. 19 shows an example of the PPG PCB 1900 (e.g., comprising one or more PPG sensors) of FIG. 16A. As shown, the PPG PCB 1900 may be secured inside of a front pad 1902 (e.g., the front pad 1602 of FIG. 16A) and may be in contact with a facepad PCB (e.g., the facepad PCB 1604 of FIG. 16A) at 1904. This PPG PCB 1900 may include a PPG sensor, for example, on the side that faces a user's forehead. In examples, the PPG information may be obtained optically via the PPG sensor and be used to determine blood volume changes in the microvascular bed of a facial tissue. For instance, the PPG sensor may include a photodiode and the PPG information may be obtained by illuminating the user's skin using a pulse oximeter (e.g., LEDs) and measuring light absorption changes via the photodiode. The PPG information thus obtained may be transmitted to the facepad PCB (e.g., the facepad PCB 1604 of FIG. 16A) and/or other circuits of the biosensing system.

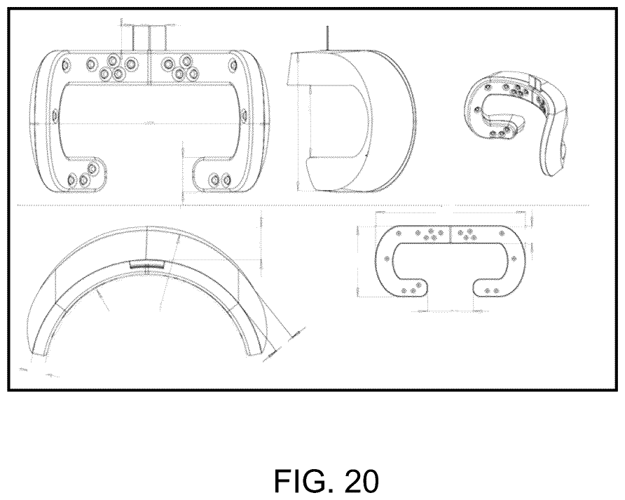

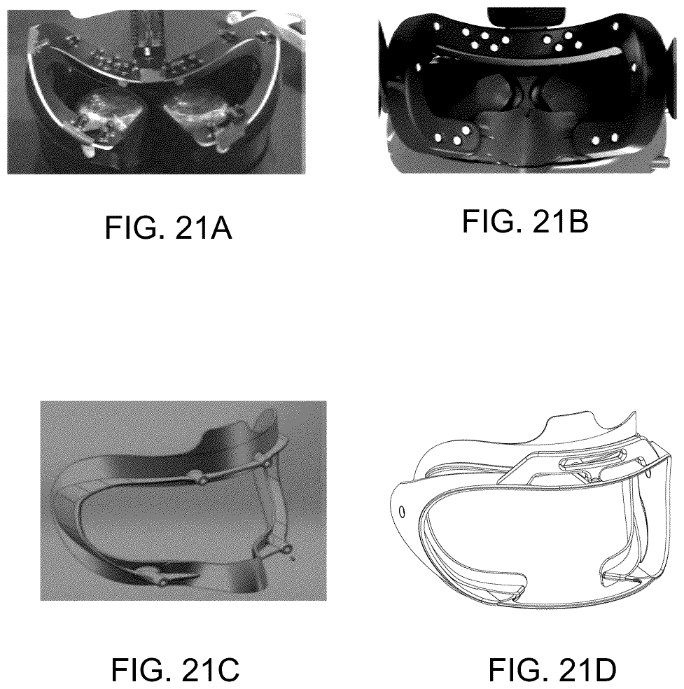

Reverting to FIGS. 16A and 16B, the gasket 1608 (e.g., a layer made of rigid plastic) may be configured to hold the front pad 1602, the facepad PCB 1604, and/or the mounting pad 1606 in place and/or to provide protection to those components. The mounting pad 1606 (e.g., a single-layer pad made of memory foam) in front of the gasket 1608 may serve as a cushion for the facepad PCB 1604 and/or to facilitate the attachment of the facepad PCB 1604 to the facepad subsystem. For instance, the mounting pad 1606 may be compressible (e.g., as a characteristic of the material from which the mounting pad is made) and as such it may provide additional pressure and/or strain relief for the facepad PCB 1604 and a user's face. Further, since the mounting pad 1606 may be located between one or more facepad electrodes (e.g., the electrodes of the facepad PCB 1604) and the face gasket 1608, the mounting pad 1606 may serve as a spring behind the electrodes to increase the comfort level of the facepad subsystem (e.g., by providing pressure relief for the electrodes in contact with the user's face). The mounting pad 1606 may also operate as an electromagnetic shield for the facepad PCB 1604 to insulate the PCB from the influence of environmental electrical noise as well as electrical noise generated by a connected device such as an attached HMD. Additional examples of the facepad subsystem and/or the various layers described herein may be illustrated by FIGS. 20 and 21A-21D.

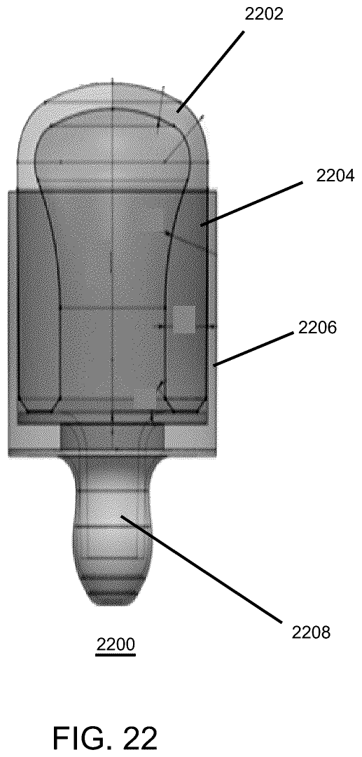

FIG. 22 illustrates an example 2200 of the facepad electrodes described herein (e.g., the electrodes 1804 shown in FIGS. 18A-18F) that is designed to be comfortable, replaceable, and/or capable of maintaining close contact with a user's face during use (e.g., regardless of whether the user's head is stationary or moving). As shown, the electrodes 2200 may include a conductive surface 2202, a wall 2204, an electrode casing 2206 (e.g., a cylindrical casing), and/or a snap connector 2208 (e.g., a male snap connector). The conductive surface 2202 may be made of a conductive polymer material. The wall 2204 may also be made of a polymer material and, together with the conductive surface 2202, may form a hollow center. The conductive surface 2202 and/or the wall 2204 may be enclosed within the casing 2206, with at least a portion of the conductive surface 2202 extending beyond the top of the casing 2206 to contact a user's face during use. The casing 2206 may be made of a rigid conductive material and may form a part of a conductive path for the signals collected by the conductive surface 2202. The bottom of the casing 2206 may be connected to the snap connector 2208 to form a base (e.g., the base may also be a part of the conductive path for the signals collected by the electrode 2200), allowing the electrode 2200 to be snapped into or out of a female connection point (e.g., the female snap connectors 1802 on the facepad PCB shown in FIGS. 18A-18F). Having the ability to snap the electrode 2200 in and out of the embedded flexible facepad PCB may render the electrode 2200 replaceable and/or recyclable, thus reducing the costs associated with making, using, and/or maintaining the biosensing system described herein.

In examples, the conductive surface 2202 may be made of silicone with conductive additive and/or EPDM rubber (ethylene propylene diene monomer rubber). Using these soft, flexible materials for the conductive surface 2202 may result in the conductive surface being gentler and more comfortable to a user's face when pressure is applied (e.g., similar to the use of a stylus on a touch screen device). This may contrast with using a rigid metal material for the conductive surface 2202, which may concentrate the force of connection on a smaller surface area, making the device less comfortable to the user's face. The hollow cavity surrounded by the conductive surface 2202 and the wall 2204 may encourage the conductive surface 2202 to compress inwards towards the base of the electrode 2200 when the device is in use. This way, a larger surface area of the conductive surface 2202 may be in contact with the user's face, allowing for an increased flow of electrons into the electrode and improving the quality of signal collection.

The conductive surface 2202 may be thinner than the wall 2204 so that the conductive surface 2202 may feel softer on the user's skin and may deform more easily under pressure. Further, making the conductive surface 2202 thinner than the wall 2204 may encourage the conductive surface 2202 to bend more readily than other parts of the electrode 2200 when pressure is applied. On the other hand, making the wall 2204 thicker (e.g., and more rigid) may give the polymer insert more structure within the casing 2206 and prevent the conductive surface 2202 from flexing away from the wall 2204 or the base of the electrode 2200, thus securing the conductive path that may run between the user's face and the facepad PCB via the base of the electrode 2200.

The systems and instrumentalities described herein may operate together with and/or be facilitated by machine-readable instructions (e.g., software and/or firmware) that may be stored in one or more memory devices and executable by one or more processors (e.g., CPUs, GPUs, MPUs, etc.). For example, when executed, these instructions (e.g., as a part of the firmware of the one or more PCBs described herein) may allow a user to initialize the systems or instrumentalities and/or to configure the settings of the systems or instrumentalities. The instructions may also cause the data collected by the systems or instrumentalities to be transmitted to a receiving device, for example, via a wired or wireless communication link (e.g., via a WiFi connection). The instructions may also allow users to initiate data collection sessions, troubleshoot and adjust sensor settings, visualize collected data alongside HMD content, integrate additional data streams, send data to other programs or services, etc. The data transmitted (e.g., to a receiving device or program) by the systems and instrumentalities described herein may be arranged in an array (e.g., a 2D array) comprising raw signal values in bytes. The receiving device or program may interpret the data array and render the data for visualizations relevant to the specific data type. For example, EEG data may be displayed as a timeseries, an FFT plot, a head plot, etc. When executed, the instructions described herein may also create one or more APIs for transmitting biometric data and/or metadata about the certain system and device configurations to a receiving API written in common programming languages such as Python, C++, C#, R, Java, MATLAB, and Julia.

A processing device as described herein may include a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. A communication circuit and/or communication link described herein may include a local area network (LAN), a wide area network (WAN), the Internet, a wireless data network (e.g., a Wi-Fi, 3G, 4G/LTE, or 5G network). A memory device described herein may include a storage medium configured to store machine-readable instructions that, when executed, cause a processing device to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. A memory device described herein may also include a mass storage device such as a magnetic disk (e.g., a hard drive), a removable disk, a magneto-optical disk, a CD-ROM or DVD-ROM disk, etc.

It should be noted even if some operations or functions are depicted and described herein with a specific order, these operations or functions may occur in various other orders, concurrently, and/or with other operations or functions not presented or described herein. Not all operations that the biosensing system is capable of performing are depicted and described herein, and not all illustrated operations are required to be performed by the biosensing system.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

What is claimed:

1. A wearable headset system, comprising:
a display configured to provide visual stimuli to a user;
a speaker configured to provide audio stimuli to the user;
a strap subsystem configured to secure the wearable headset system onto the user's head and position multiple physiological sensors at multiple locations on the user's scalp, wherein the strap subsystem includes:
a midline rail configured to run along a top of the user's scalp, from front to back, wherein a first set of physiological sensors are positioned on the midline rail, the first set of physiological sensors configured to collect a first set of biometric signals from the user's scalp; and
an occipital section configured to engage back of the user's scalp, wherein a second set of physiological sensors are positioned on the occipital section, the second set of physiological sensors configured to collect a second set of biometric signals from the user's scalp; and
a facepad subsystem configured to engage the user's face, wherein a third set of physiological sensors are positioned on the facepad subsystem, the third set of physiological sensors configured to collect a third set of biometric signals from the user's face, further wherein the facepad subsystem includes a front pad, the front pad including one or more cutouts for the third set of physiological sensors to pass through the front pad and contact the user's face;
wherein:
multiple types of biometric signals are collected from the user's head via the first set of physiological sensors, the second set of physiological sensors, and the third set of physiological sensors;
the user's physiological reactions to the visual stimuli provided via the display and/or the audio stimuli provided via the speaker are determined based on the multiple types of biometric signals collected from the user's head; and
the wearable headset system provides a neurofeedback loop for measuring, analyzing, and altering the user's physical and/or emotional states or conditions.

2. The wearable headset system of claim 1, wherein determination of the user's physiological reactions to the visual stimuli provided via the display and/or the audio stimuli provided via the speaker based on the multiple types of biometric signals collected from the user's head includes determination of physiological changes of the user's brain, eyes, skin, heart, and/or muscles in response to the visual stimuli and/or the audio stimuli.

3. The wearable headset system of claim 1, wherein the multiple types of biometric signals collected from the user's head via the first set of physiological sensors, the second set of physiological sensors, and the third set of physiological sensors include EEG (Electroencephalography) signals, EOG (Electrooculography) signals, EMG (Electromyography) signals, EDA (Electrodermal activity) signals, and/or PPG (Photoplethysmography) signals.

4. The wearable headset system of claim 1, further comprising a guide arm configured to connect the strap subsystem to one or more other parts of the wearable headset system, the guide arm extendable along a midline direction of the user's head to make the strap subsystem adjustable to different head sizes and/or different head shapes.

5. The wearable headset system of claim 1, further comprising a rear adjuster configured to allow adjustment of the strap subsystem to fit the user's head size and to adjust pressure applied to the user's scalp by the strap subsystem.

6. The wearable headset system of claim 1, wherein:
the first set of physiological sensors are positioned on the midline rail to be aligned with midline areas Fz, Cz, Pz, and Oz of the user's scalp when the wearable headset system is worn by the user; and
the second set of physiological sensors are positioned on the occipital section to be aligned with occipital areas P3, P4, PO7, and PO8 of the user's scalp when the wearable headset system is worn by the user.

7. The wearable headset system of claim 1, wherein the third set of biometric signals collected from the user's face includes a PPG signal, a bias signal, a SRB2 signal, an EDA signal, an EEG signal, an EMG signal, and an EOG signal.

8. The wearable headset system of claim 1, wherein the strap subsystem further comprises one or more circuit boards housed along the midline rail, the one or more circuit boards configured to collect and process the biometric signals collected from the user's head.

9. The wearable headset system of claim 8, wherein the processing of the biometric signals by the one or more circuit boards includes analog-to-digital signal conversion, noise and/or interference removal, timestamp tagging, and/or signal organization.

10. The wearable headset system of claim 9, wherein:
the strap subsystem comprises multiple circuit boards;
the multiple circuit boards are distributed along the midline rail;
the multiple circuit boards are connected via one or more communication cables housed along the midline rail;
further wherein distribution of the multiple circuits along the midline rail enables the strap subsystem to be aligned with shape of the user's head, thereby increasing sensitivity and accuracy of biometric signal collection while improving comfort and adjustability of the wearable headset system.

11. The wearable headset system of claim 1, wherein the wearable headset system operates as a standalone system to provide the neurofeedback loop for measuring, analyzing, and altering the user's physical and/or emotional state or conditions, further wherein the wearable headset system includes a processing unit configured to provide a single system clock for collection of the multiple types of biometric signals from the user's head and unification of the multiple types of biometric signals for neurofeedback application.

12. The wearable headset system of claim 1, wherein the visual stimuli provided via the display to the user includes AR content and/or VR content.

13. The wearable headset system of claim 1, wherein the display is integrated and/or removable from the wearable headset system.

14. The wearable headset system of claim 1, wherein the user's physiological reactions to the visual stimuli provided via the display and/or the audio stimuli provided via the speaker are used to evaluate the user's emotional responses to the visual stimuli and/or the audio stimuli.

15. The wearable headset system of claim 14, wherein the visual stimuli provided via the display and/or the audio stimuli provided via the speaker are dynamically adapted based on the user's physiological reactions and/or the user's emotional responses.

16. The wearable headset system of claim 14, wherein an AR/VR experience is generated or enhanced based on the user's physiological reactions and/or the user's emotional responses, wherein the AR/VR experience enables the user to receive neurofeedback stimulation and improve the user's skills in an environment.

17. The wearable headset system of claim 14, wherein a computing device is controlled based on the user's physiological reactions and/or the user's emotional responses.

18. The wearable headset system of claim 1, wherein operation of the wearable headset system is controlled and/or configured based on control information received from an external computing device, the control information including:
operating parameters of the first set of physiological sensors, the second set of physiological sensors, and/or the third set of physiological sensors; and/or
a destination to transmit the collected biometric signals.

19. The wearable headset system of claim 1, wherein operation of the wearable headset system is adjustable via one or more APIs to: initiate data collection sessions, troubleshoot or adjust sensor settings, visualize collected data alongside display content, integrate additional data streams, and/or send data to external programs or services.

20. The wearable headset system of claim 1, wherein the speaker includes over-ear headphones.
